(19) United States
(12) Patent Application Publication
(10) Pub. No.: US 2025/0213513 A1
(43) Pub. Date: Jul. 3, 2025

(54) USES AND APPLICATIONS OF DICARBOXYLIC ACIDS

(71) Applicant: JEMYLL LIMITED, London (GB)

(72) Inventor: Lidia CASTAGNETO GISSEY, Roma (IT)

(73) Assignee: JEMYLL LIMITED, London (GB)

(21) Appl. No.: 19/009,629

(22) Filed: Jan. 3, 2025

Related U.S. Application Data
(62) Division of application No. 16/956,859, filed on Jun. 22, 2020, now abandoned.

(30) Foreign Application Priority Data
Dec. 22, 2017 (IT) 102017000149108
Dec. 22, 2017 (IT) 102017000149147

(51) Int. Cl. A61K 31/194; A01G 7/00; A01G 22/20; A01G 33/00; A23K 10/30; A23K 20/105; A23K 50/10; A23K 50/75; A23L 7/10; A23L 13/00; A23L 15/00; A61K 9/00

(52) U.S. Cl. CPC A61K 31/194; A01G 7/00; A01G 22/20; A01G 33/00; A23K 10/30; A23K 20/105; A23K 50/10; A23K 50/75; A23L 7/10; A23L 13/00; A23L 15/00; A61K 9/0056; A23V 2002/00

(57) ABSTRACT

The present invention refers to new methods for the production of food, and/or drinks, and/or dietary supplements with a low content of cholesterol and/or with a high content of dicarboxylic acids. The present invention refers also to foodstuffs and/or drinks and/or dietary supplements that can be obtained with these methods and to their utilization, for instance in the prevention and treatment of diseases where carbohydrates and/or lipids are not correctly metabolized or in any of the pathologic states associated with insulin resistance, diabetes, hyperlipidemia, obesity, and Alzheimer disease. Specifically, the present invention refers to methods to produce low-cholesterol eggs and to methods to produce plants both terrestrial and aquatic plants and/or algae for food use, and/or dietary supplements and/or drinks enriched with dicarboxylic acids.

### TECHNICAL FIELD OF THE INVENTION

The present invention refers to new methods for the production of food, and/or drinks, and/or dietary supplements with a low content of cholesterol and/or with a high content of dicarboxylic acids. The present invention refers also to foodstuffs and/or drinks and/or dietary supplements that can be obtained with these methods and to their utilization, for instance in the prevention and treatment of diseases where carbohydrates and/or lipids are not correctly metabolized or in any of the pathologic states associated with insulin resistance, diabetes, hyperlipidemia, obesity, and Alzheimer disease.

Specifically, the present invention refers to methods to produce low-cholesterol eggs and to methods to produce plants both terrestrial and aquatic plants and/or algae for food use, and/or dietary supplements and/or drinks enriched with dicarboxylic acids.

### STATE OF THE ART

Dicarboxylic acids (DAs) with medium and long carbon atom chains are naturally occurring substances present in both plants and animals that derive from the ω-oxidation of fatty acids.

In vascular plants, DAs are components of natural protective polymers, cutin and suberin, support biopolyesters that waterproof the leaves and fruits, regulating the flow of nutrients and minimizing the harmful impact of pathogens.

Dihydroxy C16 fatty acids, 18-hydroxy-9,10-epoxy C18 fatty acids and trihydroxy C18 fatty acids are the major components of cutin, while suberin is mainly composed of ω-hydroxy fatty acids and C16-C18 dicarboxylic acids. Dicarboxylic acids are β-oxidized in specialized plant peroxisomes (glyoxysomes), where the glyoxylate cycle, whose intermediate substrates derive from the degradation of reserve or structural lipids, takes place.

Even-numbered dicarboxylic acids are suitable energy substrate, with chemical and metabolic characteristics intermediate between glucose and fatty acids. In fact, they are β-oxidized like fatty acids but, like glucose, their salts are soluble in water, thanks to their short-to-medium chains and the presence of two terminal carboxylic groups that form hydrogen bonds with water. Their end product of β-oxidation are acetyl-CoA and succinic acid, which enters the tricarboxylic acid (TCA) cycle, also known as citric acid cycle or Krebs cycle. Amino acids and some chained fatty acids can be metabolized into Krebs intermediates and enter the cycle at several points.

In animals and humans, medium-chain DAs are even-numbered, with a chain length from 6 to 12 carbon atoms, including adipic (C6), suberic (C8), sebacic (C10) and dodecanedioic (C12) acids, are efficiently metabolized. These DAs derive from the β-oxidation of longer chain DAs, which are formed by 0-oxidation from free fatty acids of the same chain length inside the microsomial membranes, or they originate from a vegetable-rich diet. However, a direct ω-oxidation of a medium-chain fatty acid, lauric acid, to dodecanedioic acid has been also demonstrated. β-Oxidation of DAs takes place in both mitochondria and peroxisomes. Four different mitochondrial pathways for DA transportation have been shown, and they include an electrophoretic transport via an inner membrane anion channel, a passive diffusion, a tributyltin-mediated transport and a transportation via the dicarboxylate carrier, which operates for short-chain DAs, such as oxalate, malonate and succinate. This transportation is carnitine independent, i.e. it does not require the carnitine shuttle, carnitine palmitoyltransferase 1, carnitine palmitoyltransferase 2 and carnitine acetyltransferase. However, a previous study demonstrated that sebacic and dodecanedioic acids consume carnitine when entering the mitochondria.

In any case, once in the mitochondria, DAs follow the same fate as free fatty acids, being degraded to acetyl-CoA through β-oxidation. A characteristic of DAs, however, is that they produce succinyl-CoA at the end of the 1-oxidation process.

Aim of the present invention is to provide new methods for the production of foodstuffs with a low content of cholesterol and/or with a high content of dicarboxylic acids. The present invention refers also to food products obtainable with these methods and to their uses.

### SUMMARY OF THE INVENTION

The present invention is based on the experiments here reported, more in details the inventors surprisingly discovered that laying hens under a diet enriched with dicarboxylic acids, and specifically with C12, produce eggs with a drastically lower content of cholesterol and with a higher weight.

Another important discovery on which the present invention is based, it is that dicarboxylic acids, and specifically C12, can be used for animal farming and for both terrestrial and aquatic plants and algae culture purposes in order to obtain products enriched with dicarboxylic acids. In particular, dicarboxylic acids will be used in hydroponic cultures, but also dissolved under form of salts or added directly to the soil for culture purposes.

Objects of the present invention are:

A method to produce eggs or milk with a lower cholesterol content including a step where an animal that produces eggs or milk is fed a diet enriched with dicarboxylic acids, specifically C12.

The use of dicarboxylic acids, and in particular of dodecanedioic acid C12, in the diet of laying animals in order to obtain eggs with a lower cholesterol content and/or a higher weight.

The eggs obtained with the method here described are object of the present invention, specifically eggs with a low cholesterol content. In the present description, low cholesterol content means a cholesterol content in the yolk lower than 200 mg, more preferably lower than 100 mg.

A method for the production of animal origin matter for food use with a high content of dicarboxylic acids that includes a step in which a farm animal is fed a diet enriched with dicarboxylic acids. Specifically, the farm animals will be cattle, ovine, swine, birds, for instance hen.

A method for the production of vegetable matter for food use with a high content of dicarboxylic acids that includes a step in aerial plants o algae from which the above mentioned vegetable matter is cultivated in the presence of dicarboxylic acids.

In the present description a vegetable or animal matter with a high content of dicarboxylic acids means a matter, such as for example milk, egg, and flours, with a concentration of dicarboxylic acids higher than 1 mg, but preferably higher than 100 mg per gram, of the above mentioned matter.

The products for food use that are obtained with the methods reported in the present invention, as well as their use to prevent and/or treat diseases in which carbohydrates and/or lipids are metabolized incorrectly—and specifically rare diseases including for instance disorders of the glycolysis, such as a deficit of triphosphate isomerase, or alterations of the lipid metabolism, such as the deficit of triosephosphate isomerase, or alterations of the lipid metabolism, such as deficit of acyl-CoA dehydrogenase—and furthermore in the presence of insulin resistance, diabetes, hyperlipidemia, non-alcoholic fatty liver disease (NAFLD), non-alcoholic steato-hepatitis (NASH), obesity, and Alzeheimer disease. These products can be also used to increase physical performance, such as, for instance, in athletes or in any healthy individual who wants to do physical exercise.

The preferred characteristics of the present invention are object of the related claims.

Other advantages, features and mode of use of the present invention will become evident in the following detailed description of some embodiments, reported as non-limiting examples.

### BRIEF DESCRIPTION OF THE FIGURES

Thereafter, we will refer to the attached figures of the drawing, where:

FIGS. 1A and 1B: Eggs from laying hens fed a standard diet or a diet enriched with C12 (10%);

FIG. 2: Weight of eggs from laying hens under a standard diet and under a diet enriched with C12. Egg mass=egg weight.

FIG. 3: Cholesterol content per 100 g of egg from laying eggs under either a standard or a C12 enriched diet (10%).

FIG. 4: C12 concentration in oat grains at increasing levels of C12 in the irrigation water.

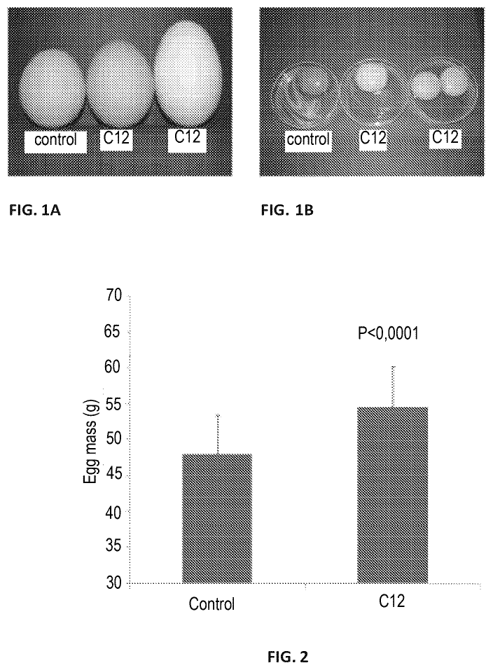

### DETAILED DESCRIPTION OF THE INVENTION

The present invention refers to a method to produce eggs or milk with low cholesterol content that includes a step in an animal producing eggs or milk fed a diet enriched with dicarboxylic acids.

In the present invention, “Enriched with dicarboxylic acids” means that dicarboxylic acids or their salts or compounds are added to the normal nourishment for each single animal or plant.

According to one embodiment, dicarboxylic acids have a number of carbon atoms between 6 and 18, for instance they can be chosen among adipic acid (C6), suberic acid (C8), azelaic acid (C9), sebacic acid (C10), dodecanedoic acid (C12) or their mixtures. Preferably, C12 will be used.

According to one embodiment, in order to obtain eggs with a low cholesterol content and/or increase the egg weight, a laying hen will be fed a standard diet, such as wheat and/or soybeans, with the addition of at least 5%, but preferably at least 10% (weight to weight) of dicarboxylic acids.

The present invention refers also to a method to produce vegetable matter for food use enriched with dicarboxylic acids that includes a step in which aerial plants or algae, from which the above-mentioned vegetable matter is obtained, have grown in the presence of dicarboxylic acids.

As specified above, those dicarboxylic acids utilized in growing the above mentioned plants and algae can be added to the soil or to the irrigation water; they will have preferably a number of carbon atoms ranging from 6 to 18, they can be chosen for instance among adipic acid (C6), suberic acid (C8), azelaic acid (C9), sebacic acid (C10), dodecanedioic acid (C129 or their mixtures. More preferably, C12 will be used.

The amount of dicarboxylic acids supplied with the irrigation water will be preferably that allowing a concentration between 1 and 10 g/L.

According to one embodiment, the above-mentioned plants are cereals, and specifically barley or oats.

According to one embodiment, the above-mentioned plants have grown in hydroponic cultures.

The present invention refers also to a method to produce matter of animal origin for food use enriched with dicarboxylic acids that includes a step in which a farm animal is fed a diet enriched with dicarboxylic acids.

Dicarboxylic acids with a number of carbon atoms between 6 and 18—for instance they are chosen among adipic acid (C6), suberic acid (C8), azelaic acid (C9); sebacic acid (C10), dodecanedoic acid (C12) or their mixtures—can be used in the above-mentioned breading method. More preferably, C12 will be used.

The amount of dicarboxylic acids supplied will preferably range between 1 and 100 g per day.

In all the embodiments here described, dicarboxylic acids can be administered to the animals both orally and by injection.

Plants and algae or their derivatives, such as but not only cereals and flours, and thus snacks, pasta, bread, etc., or fruits and their derivatives, such as but not only marmalades, etc., rich of dicarboxylic acids, which can be obtained with the method described here can be utilized for food use.

Snack means for instance sweets, chocolate bars, etc. C12 and other dicarboxylic acids that are added to the food for animals, including fish, can be used as food—under form of milk and milk products such as ricotta cheese, different types of cheese, mozzarella cheese, yogurt, etc., or under form of eggs and egg products—for human or for pet use.

Therefore, both vegetal (such as flours, drinks, etc.) and animal (such as eggs, milk, meat, etc.) products enriched with DAs can be used to produce foodstuffs.

Furthermore, dicarboxylic acids or their salts or derivatives, such as but not only triglycerides, esters and amino acids, can be added—although they can derive from other production systems—to drinks, snacks and other foodstuffs or can be administered for enteral nutrition.

The indications for the use of DAs are those to supply a nutrient that can be a total or partial substitute of carbohydrates and/or fatty acids, as in the case of hypoglucidic or hyperproteic or hyperlipidic diets or mixed diets, but also those to supply a nutrient to prevent and/or treat rare diseases in which carbohydrates and/or lipids cannot be adequately utilized or in the presence of insulin resistance, diabetes, hyperlipidemia, obesity, Alzheimer disease, different types of metabolic alterations, etc.

Another indication is that of increasing the physical performance of athletes or any individual who wants to perform either competitive or non-competitive physical activity. In this patent we describe two examples of application.

Another direct utilization of C12 and of other dicarboxylic acids, even if they are already commercialized or chemically purified but for human use, is their addition alone or together with mineral salts, caffeine, amino acids, carnitine, fructose, glucose or other energy substrates or sweeteners to still or sparkling energy drinks, other than the preparation of substitute meals for obesity, diabetes, metabolic syndrome, insulin resistance, kidney failure, etc., other than their use for rare diseases in which there is an insufficient utilization of carbohydrates and/or lipids.

The examples reported in the following further illustrate, in a non-limitative way, the invention.

### Examples of Realization and Experimental Data

### Case 1: Low Cholesterol Eggs

### Introduction

Eggs are a major human foodstuff as they provide most of the nutrition principles as suggested by all recommended daily allowance.

However, the variability in the quality and nutritional values of eggs has a significant impact on consumers' health. In fact, 30% of egg yolk content is made of lipids with a high cholesterol content. Egg consumption is also believed to raise the risk of cardiovascular disease by increasing blood cholesterol levels in hyper-responders and diabetics, as well as persons with elevated LDL-cholesterol levels and heart disease, who should all limit their dietary cholesterol intake to remain healthy.

Until recently, reducing dietary cholesterol has been a part of the American Heart Association (AHA) and American College of Cardiology (ACC) guidelines on lifestyle management, despite unconvincing evidence to support the recommendation. Rather than strictly limiting cholesterol intake, the AHA and ACC guidelines now recommend dietary patterns that emphasize fruits, vegetables, whole grains, low-fat dairy products, poultry, fish, and nuts as an approach to favorably altering blood lipid levels. Although the 2015-2020 Dietary Guidelines for Americans have removed the recommendation of limiting cholesterol intake to no more than 300 mg per day, they advise that individuals should eat as little dietary cholesterol as possible while following a healthy diet.

Other cardiovascular nutrition guidelines continue to include a recommendation to limit dietary cholesterol to less than 200 mg per day.

Limiting consumption of saturated fat automatically tends to limit dietary cholesterol. Since eggs are a rich source of dietary cholesterol, typically containing 141-234 mg per egg, also egg consumption is limited.

A separate case is that of diabetic individuals. Meta-analyses of prospective cohort studies of diabetes show that the consumption of one or more eggs per day is linked with about 50-70% increased risk of cardiovascular events compared to those who seldom eat eggs.

Therefore, restricting the number of eggs consumed daily is useful in particular in subjects with diabetes, but also in those who have hyperlipidemia and/or an increased risk of cardiovascular diseases (CVD).

### Methods

Thirty Hy-Line Brown laying hens, 18 weeks of age, were free range-raised under natural light and dark cycle. The hens were divided into 2 groups and housed in different indoor pens (0.15 m²/bird) equipped with feeders and drinkers, with access to open air runs (3 m²/bird).

The diets were based on wheat and soybean meal with added dodecanedioic acid (C12) at 10% in the DAs diet, while they were free of dodecanedioic acid in the control diet. The diets were isocaloric and isonitrogenous containing 17.0% of crude protein (CP) and 2,680 kcal of metabolizable energy/kg of diet, in fact they were designed to meet or exceed the nutrient requirements for laying hens. The experimental diet was fed to the animals for 12 weeks. Feed and water were provided ad libitum throughout the entire trial.

Hens' mortality or morbidity were recorded if occurred. Eggs were collected daily and egg production was calculated on per hen and per day basis. Eggs with any adhering manure were classified as dirty, and the percentage was then calculated.

Eggs were analyzed for their interior and exterior physical quality, and were also examined for shell quality by specific gravity. Shell thickness (with shell membrane) of the eggs (10% of the daily egg produced) was measured by micrometer. Shell thickness was a mean value of measurements at 3 locations on the eggs (air cell; equator; and, sharp end). The breaking strength of uncracked eggs was determined with a testing machine (model 1140, Instron Ltd., Bucks, UK). Egg components (albumen; yolk; and, shell) were measured by weekly breakouts on two eggs per replicate pen and expressed as percentage of egg weight.

Egg yolk color was scored using the 15-point scale (color scale from 15, dark orange to 1, light pale) of the DSM yolk color fan (DSM Nutritional Products Ltd., Basel, Switzerland).

The yolk cholesterol concentrations were determined by sampling weekly egg yolks (1 g) saponified with 20 ml of 33% ethanolic KOH in tightly-capped tubes placed in a 60° C. water bath for 1 hour. The mixture was then cooled in ice water, and 5 ml of distilled water was added. Cholesterol in unsaponifiable fractions was extracted twice with 5 ml of hexane. The resulting aliquot of hexane containing cholesterol was dried under nitrogen, re-dissolved in 5 ml of hexane, and injected into a gas chromatograph (Hewlett Packard, Palo Alto, CA, Palo Alto, CA, USA).

5 α-cholestane (Sigma-Aldrich) was used as an internal standard. A split inlet (using a split ratio of 100:1) was used to inject samples into a capillary column (HP-5, Agilent, Steven, CA, USA; 30 μm×0.53 mm-0.5 μm). Temperature conditions were as follows: ramped oven temperature of 270° C. isothermal; detector temperature of 300° C.; and, inlet temperature of 210° C. The gas carrier was N₂ kept at a constant flow rate of 1.0 ml/min.

Ten staff members randomly and blindly tested the digestibility and palatability of the two types of eggs in triplicates.

Data were analyzed using the statistical package SPSS 13. Comparisons were made by the Mann Whitney U test since data were not normally distributed.

### Results

No hen mortality or morbidity was observed. Data regarding the effects of the different dietary regimes on egg productivity and quality parameters as well as on the amount of yolk cholesterol content are reported in Table 1.

TABLE 1
Eggs characteristics under standard diet or diet enriched
with dodecanedioic (C12) acid. The content of C12
in a yolk ranged between 0.35 and 2.1 mg.

| | Standard diet | C12 diet | P |
|---|---|---|---|
| Egg-laying rate (%) | 88.20 ± 6.45 | 90.30 ± 4.99 | 0.25 |
| Dirty eggs (%) | 0.43 ± 0.14 | 0.38 ± 0.14 | 0.25 |
| Shell thickness (mm × 10⁻²) | 0.36 ± 0.05 | 0.38 ± 0.037 | 0.36 |
| Egg mass (g/hen per day) | 47.93 ± 5.53 | 54.56 ± 5.66 | <0.0001 |
| Cholesterol content | 330.25 ± 67.17 | 87.00 ± 14.27 | <0.0001 |

Digestibility and palatability of the two types of eggs were indistinguishable from one another.

### Conclusions

A 10% C12 enriched diet was able to drastically reduce cholesterol content of eggs while significantly increasing egg mass. The other characteristics of the eggs were not affected by C12 intake, except for the lighter color of the yolk and the occasionally double yolk.

In the United States a light-coloured yellow yolk is generally preferred by consumers.

In Europe a real North-South divide can be observed. While the northerners prefer pale yellow yolks, the preference of consumers for golden-yellow yolks grows as we go further south. On the shores of the Mediterranean, only bright, orange-red yolks stand a chance of reaching the plate. In contrast, UK consumers prefer paler yolk colors and reject very dark yolks.

### Case 2: Dodecanedioic Acid in Grains

Hydroponic barley and oat grass was growth in fodder sprouting chambers at 21.5 to 23.5° C., with humidity of 65±5% and water temperature between 20° and 22° C.

Grains were soaked in water until fully saturated, then drained and placed in trays or troughs, to facilitate sprouting, for 5 to 8 days. The grains were kept moist during this period. Grains were initially washed with a sterilizing solution to help minimize the risk of mold. Grass were provided with 1000 lux grains from day 3.

C12 (0.1% as acid, 14.9% as potassium salt, 60% as sodium salt, and 25% as calcium salt) was added to the water medium in different concentrations, varying from 0%, to 2.5%, 5%, 10%, 15% and 20%.

A dry matter recovery of over 85-90% was achieved. The concentration of C12 in oat grains increased as the amount of C12 in the water raised plateauing at 10 g/L of Cl₂, as shown in FIG. 4.

We measured grain composition at 10 g C12 in 1 L of water. The composition of barley and oat grains is reported in Table 2.

TABLE 2
Composition of barley and oat grains (10 g C12/L of water).

| | Barley grain | | Oat grain | |
|---|---|---|---|---|
| | Control | 10% C12 | Control | 10% C12 |
| Dry matter (%) | 86.00 ± 1.58 | 90.40 ± 1.14 | 92.60 ± 3.58 | 90.00 ± 5.70 |
| Starch % | 57.60 ± 3.05 | 44.40 ± 3.51 | 40.46 ± 9.18 | 31.80 ± 8.11 |
| Crude Protein % | 14.06 ± 1.44 | 16.20 ± 0.62 | 12.80 ± 3.29 | 18.70 ± 2.28 |
| Crude Fibre % | 4.60 ± 0.66 | 4.42 ± 0.38 | 19.34 ± 6.57 | 23.40 ± 4.04 |
| C12% | 0 | 3.04 ± 0.23 | 0 | 3.12 ± 0.47 |
| Ash % | 2.56 ± 0.17 | 3.40 ± 0.48 | 4.16 ± 0.50 | 4.06 ± 0.59 |

We conclude that C12 is used by barley and oat as an effective energy substrate and it is recovered in good amounts in grains.

The present invention was described with the reference to some preferred embodies. However, it is clear that other embodies forms, referred to the same invention nucleus, can exist as defined within the protection of the claims reported thereafter.

1.-23. (canceled)

24. A method for preventing and/or treating a disease wherein carbohydrates and/or lipids are not properly metabolized in a subject comprising administering to said subject a food product obtained by a method for the production of eggs or milk with a reduced cholesterol content comprising a step wherein an animal producing egg or milk is fed with a diet enriched with dicarboxylic acids.

25. The method according to claim 24, wherein said dicarboxylic acids are selected from adipic (C6), suberic (C8), azelaic (C9), sebacic (C10), and dodecanoic (C12), tetradecanedioic acid (C14), hexadecanedioic (C16) and octadecanedioic (C18) acids, their salts or mixtures thereof.

26. The method according to claim 24, wherein said diet is enriched in dicarboxylic acids with the addition of at least 5%, preferably at least 10%, to the feed of said animals.

27. A method of increasing physical performance in a subject comprising administering to said subject a food product obtained by a method for the production of eggs or milk with a reduced cholesterol content comprising a step wherein an animal producing egg or milk is fed with a diet enriched with dicarboxylic acids.

28. The method according to claim 27, wherein said dicarboxylic acids are selected from adipic (C6), suberic (CS), azelaic (C9), sebacic (C10), and dodecanoic (C12), tetradecanedioic acid (C14), hexadecanedioic (C16) and octadecanedioic (C18) acids, their salts or mixtures thereof.

29. The method according to claim 27, wherein said diet is enriched in dicarboxylic acids with the addition of at least 5%, preferably at least 10%, to the feed of said animals.

30. A method for preventing and/or treating a disease wherein carbohydrates and/or lipids are not properly metabolized in a subject comprising administering to said subject a food product comprising a vegetable material obtainable by a method for the production of vegetable material for food use with a high content of dicarboxylic acids comprising a step wherein a plant and/or an alga from which said vegetal material is grown in the presence of dicarboxylic acids.

31. The method according to claim 30, wherein said dicarboxylic acids are selected from adipic (C6), suberic (C8), azelaic (C9), sebacic (C10), and dodecanoic (C12), tetradecanedioic acid (C14), hexadecanedioic (C16) and octadecanedioic (C18) acids, their salts or mixtures thereof.

32. The method according to claim 30, wherein said diet is enriched in dicarboxylic acids with the addition of at least 5%, preferably at least 10%, to the feed of said animals.

33. A method of increasing physical performance in a healthy subject comprising administering to the subject a vegetal material obtainable by a method for the production of vegetable material for food use with a high content of dicarboxylic acids comprising a step wherein a plant and/or an alga from which said vegetal material is grown in the presence of dicarboxylic acids.

34. The method according to claim 33, wherein said dicarboxylic acids are selected from adipic (C6), suberic (C8), azelaic (C9), sebacic (C10), and dodecanoic (C12), tetradecanedioic acid (C14), hexadecanedioic (C16) and octadecanedioic (C18) acids, their salts or mixtures thereof.

35. The method according to claim 33, wherein said diet is enriched in dicarboxylic acids with the addition of at least 5%, preferably at least 10%, to the feed of said animals.
